SEQUENCE LISTING (10 sequences)

SEQ ID NO: 1 - DNA, 30 bp, Artificial Sequence
  Synthetic Oligonucleotide
  misc_feature (23)..(23): S = C or G
gagcctgcttctccctctgcctstgtctct

SEQ ID NO: 2 - DNA, 29 bp, Artificial Sequence
  Synthetic Oligonucleotide
  misc_feature (2)..(2): W = A or T
  misc_feature (5)..(5): Y = C or T
  misc_feature (21), (24): R = A or G
gwccygggatcgagtcccacrtcrggctc

SEQ ID NO: 3 - DNA, 29 bp, Artificial Sequence
  Synthetic Oligonucleotide
  misc_feature (1), (9), (13), (27): Y = C or T
  misc_feature (10), (20), (28): R = A or G
  misc_feature (18)..(18): K = G or T
yctgccttyrgcycaggkcrtgatccyrg

SEQ ID NO: 4 - DNA, 29 bp, Artificial Sequence
  Synthetic Oligonucleotide
  misc_feature (11)..(11): R = A or G
  misc_feature (27), (29): W = A or T
  misc_feature (28)..(28): M = A or C
tgtctctcatraataaataaataaaawmw

SEQ ID NO: 5 - DNA, 21 bp, Artificial Sequence
  Synthetic Oligonucleotide
  misc_feature (15)..(15): Y = C or T
cctgggtggctcagyggttta

SEQ ID NO: 6 - DNA, 15 bp, Artificial Sequence
  Synthetic Oligonucleotide
tgcctctctctctct

SEQ ID NO: 7 - DNA, 41 bp, Artificial Sequence
  Synthetic Oligonucleotide
  misc_feature (3)..(3): D = A or T or G
  misc_feature (30), (31), (32): R = A or G
  misc_feature (40)..(40): W = A or T
tgdgcctcagtttcctcatctgtaaaatgrrrataataawa

SEQ ID NO: 8 - DNA, 21 bp, Artificial Sequence
  Synthetic Oligonucleotide
  misc_feature (12), (17): W = A or T
  misc_feature (14)..(14): Y = C or T
aaataaataaawtyttwaaaa

SEQ ID NO: 9 - DNA, 49 bp, Artificial Sequence
  Synthetic Oligonucleotide
  misc_feature (2), (4), (7), (8), (10), (38), (40), (46), (48): Y = C or T
gytytryyayttactagctgtgtgaccttgggcaagtyayttaacytyt

SEQ ID NO: 10 - DNA, 21 bp, Artificial Sequence
  Synthetic Oligonucleotide
  misc_feature (1), (8), (15): Y = C or T
  misc_feature (2), (9): R = A or G
  misc_feature (5)..(5): S = C or G
  misc_feature (10)..(10): K = G or T
yrctsagyrkggagyctgctt

(19) United States
(12) Patent Application Publication
(10) Pub. No.: US 2022/0333211 A1
(43) Pub. Date: Oct. 20, 2022

(54) METHODS AND COMPOSITIONS FOR CANCER DETECTION, CHARACTERIZATION OR MANAGEMENT IN COMPANION ANIMALS

(71) Applicant: PetDx, Inc., La Jolla, CA (US)

(72) Inventors: Ilya Chorny, La Jolla, CA (US); Daniel Grosu, La Jolla, CA (US)

(21) Appl. No.: 17/806,238

(22) Filed: Jun. 9, 2022

Related U.S. Application Data
(63) Continuation of application No. PCT/US2020/065337, filed on Dec. 16, 2020.
(60) Provisional application No. 62/949,920, filed on Dec. 18, 2019.

(51) Int. Cl. C12Q 1/6886

(52) U.S. Cl. CPC C12Q 1/6886; C12Q 1/6869

(57) ABSTRACT

Provided herein are methods and kits for measuring genome wide copy number aberrations, including aneuploidies, in animals, such as in dogs, for the purposes of cancer detection or characterization or management. Also provided are particular motifs for use in measuring copy number variants including aneuploidies genome-wide in animals, such as in dogs.

### CROSS REFERENCE TO RELATED APPLICATIONS

This application is a continuation application of PCT Application No. PCT/US2020/065337, filed Dec. 16, 2020, which designates the Unites States of America, and published in the English language, and which is an International Application of and claims the benefit of priority to U.S. Provisional Application No. 62/949,920, filed Dec. 18, 2019. The discloses of the above-referenced applications are herby incorporated by reference in their entireties.

### REFERENCE TO SEQUENCE LISTING

The present application is being filed along with a sequence listing in electronic format. The sequence listing is provided as a file entitled SequenceListingPETDX.003C!, created Jun. 9, 2022, which is 3.51 KB in size. The information in the electronic format of the sequence listing is incorporated herein by reference in its entirety.

### FIELD

The present disclosure relates to methods for detecting, characterizing, or managing cancer in a companion animal by analyzing the genome-wide distribution and magnitude of copy number aberrations, including aneuploidies in the animal.

### BACKGROUND

Companion animals, such as dogs and cats are enjoying longer lifespans as veterinary medicine continues to improve. However, this increased lifespan has led to a higher rate of cancers among companion animals. By some estimates, over 50% of dogs over ten years of age are going to die from a cancer-related health issue. Cats are also susceptible to a variety of cancers. Among the most common cancers in these animals are lymphoma, squamous cell carcinoma (skin cancer), mammary cancer, mast cell tumors, oral tumors, fibrosarcoma (soft tissue cancer), osteosarcoma (bone cancer), respiratory carcinoma, intestinal adenocarcinoma, and pancreatic/liver adenocarcinoma.

Certain breeds of cats are more prone to certain cancers than others. Signs and symptoms differ depending on the type and stage of the cancer. Unfortunately, detection and diagnosis of these cancers is often difficult, and invasive biopsy tests usually need to be performed to make an accurate diagnosis.

The situation is similar for dogs. Certain canine breeds are known to be susceptible to particular cancers. For example, larger dogs are more susceptible to developing osteosarcoma. German Shepherds, Golden Retrievers, Labrador Retrievers, Pointers, Boxers, English Settlers, Great Danes, Poodles, and Siberian Huskies are susceptible to developing hemangiosarcoma (HSA). HSA tends to affect large breed animals more often than smaller ones.

Copy number aberrations are a hallmark of cancer and are known to be a common biomarker for the presence of cancer in companion animals, including dogs. Genome wide methods for detecting copy number aberrations in a hypothesis free manner are expensive because of the large amount of sequencing needed to cover the whole genome, even at low coverage, and less sensitive to focal copy number aberrations on the order of the size of a gene.

Currently available techniques do not provide for a relatively inexpensive and simple amplicon based way to perform cancer detection, or characterization by determining the distribution and magnitude of copy number aberrations (CNAs) including aneuploidies in companion animals that may be associated with the presence of a wide variety of cancer types.

### SUMMARY

Described herein are methods and compositions for the detection, diagnosis, and screening of cancer in companion animals, such as in dogs.

Some embodiments provided herein relate to methods of measuring copy number aberrations in a companion animal, or methods of determining whether a companion animal is likely to have cancer. In some embodiments, the methods include obtaining circulating cell free DNA (cfDNA) in a biological sample from a companion animal; amplifying the cfDNA using a sequence specific primer that is derived from repeat elements present throughout the genome of the companion animal to obtain copies of the repeat element and adjacent genomic sequences; determining the number and distribution of the copies of amplified regions in the cfDNA; and comparing the number and distribution of copies of the amplified regions, including the adjacent genomic sequences, to one or more healthy animals or tissue samples to determine if the number and distribution of copies in the companion animal suspected of having cancer differs from the number of copies of the amplified regions in the one or more healthy animals or tissue samples, wherein a statistically significant difference indicates that the companion animal is highly likely to have cancer.

Some embodiments provided herein relate to methods for determining if a canine animal is likely to have a cancer. In some embodiments, the methods include obtaining circulating cell free DNA (cfDNA) in a biological sample from a canine containing canine genomic sequences; amplifying short interspersed nuclear element (SINE) sequences and adjacent sequences from the canine genomic sequences to determine the number and distribution of SINE sequences in the canine genomic sequences; and determining whether the companion animal is likely to have cancer based on the number and distribution of the SINE sequences.

Some embodiments provided herein relate to methods of profiling single nucleotide variant (SNV) and copy number aberration (CNA) in a single assay. In some embodiments, the methods include obtaining circulating cell free DNA (cfDNA) in a biological sample from a canine containing canine genomic sequences; contacting the sample with primers for an SNV and with primers for a CNA using SINE spike sequences; and amplifying the SINE spike sequences; thereby determining SNV and CNA in a single assay.

Some embodiments provided herein relate to kits for determining cancer in a companion animal. In some embodiments, the kits include at least one sequence specific primer for amplifying cfDNA in a biological sample from a companion animal, wherein the at least one primer amplifies SINE repeat sequences and adjacent genomic sequences; and a polymerase for amplifying the primers.

### BRIEF DESCRIPTION OF THE DRAWING

FIG. 1 is a bar chart that depicts a canine chromosomal panel showing targeted low pass sequencing to capture the number of genome wide cancer-associated copy number aberrations and aneuploidies in each canine chromosome using a nucleic acid sequence as set forth in SEQ ID NO: 1.

FIGS. 2A-2B depict copy number aberration (CNA) profiles in a first canine sample having a confirmed diagnosis of cancer in both tumor tissue (FIG. 2A) and cfDNA (FIG. 2B). FIG. 2A depicts CNA profiles found using the SINE prep method (top) and using the shallow whole genome sequencing (sWGS) method (bottom) in tissue samples. FIG. 2B depicts CNA profiles found using the SINE prep method (top) and using the sWGS method (bottom) in cfDNA samples.

FIGS. 3A-3B depict CNA profiles in a second canine sample having a confirmed diagnosis of cancer in both purported tumor tissue (FIG. 3A) and cfDNA (FIG. 3B). FIG. 3A depicts CNA profiles found using the SINE prep method (top) and the sWGS method (bottom) in tissue samples, with no CNAs detected, indicating that no CNAs were detected in the tumor cells. FIG. 3B depicts CNA profiles using the SINE prep method (top) and the sWGS method (bottom) in cfDNA samples showing the presence of CNAs in the cfDNA samples.

FIGS. 4A-4B depict CNA profiles in a third canine sample having a confirmed diagnosis of cancer in both tumor tissue (FIG. 4A) and cfDNA (FIG. 4B). FIG. 4A depicts CNA profiles using the SINE prep method (top) and the sWGS method (bottom) in tissue samples. FIG. 4B depicts CNA profiles using the SINE prep method (top) and the sWGS method (bottom) in cfDNA samples, with no CNAs detected, indicating that the cfDNA may not necessarily include the full heterogeneity of the individual's tumors.

FIGS. 5A-5C are box charts depicting the evaluation of single nucleotide variant (SNV) calls from SINE primer spiked samples. FIG. 5A depicts the total reads for SINE, and spike-in of the indicated concentrations of SNV panel primers, including 0 nM, 0.1 nM, 0.4 nM, 1.56 nM, 6.25 nM, and 25 nM. FIG. 5B depicts the reads on target for the same assays as FIG. 5A, and FIG. 5C depicts the mean target coverage (MTC).

FIGS. 6A-6B are box charts depicting evaluation of target regions covered at or above 500x (FIG. 6A) and 1000x (FIG. 6B).

FIGS. 7A-7C are box charts depicting uniformity of MTC across spike levels, including at 0.2 MTC (FIG. 7A), 0.5 MTC (FIG. 7B), and MTC (FIG. 7C).

FIGS. 8A-8D depict error metrics for various SINE spike-in concentrations, including chimera (FIG. 8A), sub rate (FIG. 8B), GC dropout (FIG. 8C), and INDEL rate (FIG. 8D).

FIGS. 9A-9B are box charts depicting artifacts observable in spike-in SNV SINE evaluations. FIG. 9A depicts aligned reads, indicating a small percentage of SINE reads overlap with SNV panel primers. FIG. 9B depicts mean insert size, which is higher for lower SINE-spike-in levels.

FIGS. 10A-10C are box charts depicting the counts of variants called with SINE spike-in, including SNV counts (FIG. 10A), insertion counts (FIG. 10B), and deletion counts (FIG. 10C).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein. All references cited herein are expressly incorporated by reference herein in their entirety and for the specific disclosure referenced herein.

Embodiments relate to methods, systems and compositions for screening companion animals for their likelihood to have cancer. In one embodiment cancer is screened for by analyzing the genome-wide levels of copy number aberrations in particular repeated elements of the companion animal genome. For example, many genomes contain nucleotide sequences that are repeated dozens, hundreds, or thousands of times. Analyzing whether a particular companion animal contains the typical number of such repeats overall and on each chromosome is useful to determine genetic variations indicating cancer in the animal. In particular, the methods may include determining the unique gene sequences adjacent to each repeated element. Determining the unique gene sequences adjacent to each repeated element allows each repeated element to be uniquely identified as part of the comparison. For example, a repeated element from the long arm of chromosome 11 in a healthy companion animal may be compared to the same repeated element from the long arm of chromosome 11 in an animal being screened for cancer since the unique adjacent sequences allow for such a comparison. By comparing individual repeated elements to one another, a determination can be made whether particular repeated elements have been amplified or deleted in the genome of the companion animal being screened for cancer. This provides a more robust and sophisticated manner of detecting copy number aberrations as compared to only determining the total number of repeated elements in a genome.

In one embodiment, the number of copies of one or more motifs derived from Short Interspersed Nuclear Element (SINE) sequences which are anchored to adjacent non-repeat regions, and which are widely distributed throughout the genome of companion animals, can be measured. This can be done by sequencing an amplicon comprising the motif and its adjacent non-repeat regions to determine the presence of the amplicon in the genome by its position. This may be predictive of whether the animal is suffering from cancer.

Analyzing copy number aberrations, including aneuploidies, of these amplicon sequences for abnormalities in number or sequence as compared to normal controls allows one to infer whether the companion animal may be suffering from a particular cancer which is having an effect on the genome-wide copy number of anchored SINE sequences; this can inform, for example, organ-of-origin or tissue-of-origin predictions for the suspected cancer; this can also inform, for example, identification of gene-specific amplification or deletion events, which can help direct treatment. Genetic and epigenetic features altered in cancers have been published, for example by Ciriello, et al. (Emerging Landscape of Oncogenic Signatures Across Human Cancers, Nature Genetics, 45, 1127-1133, (2013)) the contents of which are hereby incorporated by reference in its entirety. Accordingly, one embodiment is not only counting the number of amplicons, but also particularly identifying the location in the genome of each amplicon and determining if there is a statistically significant difference in the amplicon sequences between one or multiple presumably-normal control animal(s) and the animal suspected of having cancer; or between the observed and the expected number of amplicon sequences at multiple specific locations in the genome of the same subject.

In one embodiment, a PCR primer is used to amplify the nucleotide sequences adjacent to the SINE motif. Because the nucleotide sequences adjacent to each SINE motif sequence are generally unique in the genome, the specific SINE motif sequence, chromosome number and position may be determined along with the overall count of how many SINE motif sequences were found in the sample. For example, in a normal, healthy dog it may be discovered that a SINE motif is present on chromosome 6 with 8000 normalized copies of the SINE motif. However, a dog with osteosarcoma may be found to have 12000 normalized copies of the SINE motif sequences on chromosome 6. By determining not only the overall number of SINE motif sequences, but also their relative distribution and location in the genome, one can correlate the variations of the number of SINE motif sequences on a particular chromosome with a disease state, such as cancer.

In one embodiment, a healthy control is not needed to determine a copy number aberration in an animal suspected of having cancer. By comparing the number of copies of one or more amplicons in one or more case regions with one or more amplicons in one or more control regions, from the same animal suspected of having cancer, a determination of CNA can be made in the animal suspected of having cancer.

A variety of ways exist for determining the CNAs within a genome. In one embodiment a blood sample is taken from a companion animal by a veterinarian. Circulating free DNA (cfDNA) from the blood is obtained. The cfDNA is isolated by removing blood cells from the sample so that only cfDNA remains in the sample. If necessary, the cfDNA can be further fragmented and unique nucleotide barcodes (often called unique molecular identifiers) are added to the fragmented DNA in the cfDNA sample. However, in some embodiments, the cfDNA does not need to be fragmented because it already comprises fragmented regions of genomic DNA. The barcoded sample is then made single stranded and one or more sequence specific primers are added to the mixture. In some embodiments, the sequence specific primer is a nucleotide motif that has a DNA sequence found in SINE repeat element sequence, such as:

SEQ ID NO: 1
(GAGCCTGCTTCTCCCTCTGCCTSTGTCTCT)
SEQ ID NO: 2
(GWCCYGGGATCGAGTCCCACRTCRGGCTC)
SEQ ID NO: 3
(YCTGCCTTYRGCYCAGGKCRTGATCCYRG)
SEQ ID NO: 4
(TGTCTCTCATRAATAAATAAATAAAAWMW)
SEQ ID NO: 5
(CCTGGGTGGCTCAGYGGTTTA)
SEQ ID NO: 6
(TGCCTCTCTCTCTCT)
SEQ ID NO: 7
(TGDGCCTCAGTTTCCTCATCTGTAAAATGRRRATAATAAWA)
SEQ ID NO: 8
(AAATAAATAAAWTYTTWAAAA)
SEQ ID NO: 9
(GYTYTRYYAYTTACTAGCTGTGTGACCTTGGGCAAGTYAYTTAACYTY
T)
or
SEQ ID NO: 10
(YRCTSAGYRKGGAGYCTGCTT),

wherein D is A or T or G; M is A or C; R is A or G; W is A or T; S is C or G; Y is C or T; and K is G or T.

Polymerase is then added to the mixture so the sequence specific primer is then extended into, and preferably through, the repeat element to form a specific sequence that includes the repeated element plus additional unique nucleotides adjacent to the repeated element. After the sequence specific primer has been extended, a sample index primer that contains a unique multiplex code and a sequencing primer region along with a universal primer, is used to amplify the extended sequence. The amplified fragments include sequencing ends which are formatted to be used within a Next Generation Sequencing (NGS) system to identify the nucleotide sequences of the repeated element, such as SINE sequence, and any adjacent nucleotide sequences.

The number of amplicon sequences in the cfDNA sample, and their positions in the genomic map of the companion animal being analyzed, may then be calculated for each chromosome in the companion animal. In one embodiment, this aforementioned process is part of a QIAseq kit available from QIAGEN (Hilden, Germany).

Methods and compositions provided herein improve the detection, diagnosis, staging, screening, treatment, and management of cancer in companion animals, particularly in dogs, cats and other types of companion animals. As mentioned above, embodiments include identifying copies of repeated nucleic acid sequence elements in circulating biological fluids, such as blood. In one embodiment, the nucleic acid sequence elements are found in circulating tumor DNA in the blood.

Accordingly, some embodiments provided herein relate to methods of determining whether a companion animal is likely to have cancer. Some embodiments relate to methods of measuring copy number aberrations in a companion animal. In some embodiments, the methods include obtaining circulating cell free DNA (cfDNA) in a biological sample from a companion animal; amplifying the cfDNA using a sequence specific primer that is derived from repeat elements present throughout the genome of the companion animal to obtain copies of the repeat element and adjacent genomic sequences; determining the number and distribution of the copies of amplified regions in the cfDNA; and comparing the number and distribution of copies of the amplified regions, including the adjacent genomic sequences, to one or more healthy animals to determine if the number and distribution of copies in the companion animal suspected of having cancer differs from the number of copies of the amplified regions in the one or more healthy animals, wherein a statistically significant difference indicates that the companion animal is highly likely to have cancer.

In some embodiments, the companion animal is a dog. In some embodiments, the sequence specific primer is present in SINE sequences. In some embodiments, the sequence specific primer has a nucleotide sequence as set forth in any one of SEQ ID NOs: 1, 2, 3, 4, 5, 6, 7, 8, 9, or 10, or any combination or variant thereof. In some embodiments, the variant has a sequence identity of at least 75% to the sequence as set forth in any one of SEQ ID NOs: 1-10, such as 75%, 80%, 85%, 90%, 91%, 92%, 93,%, 94%, 95%, 96%, 97%, 98%, or 99%, or a sequence identity within a range defined by any two of the aforementioned values. In some embodiments, the sequence specific primer has a nucleotide sequence of SEQ ID NO: 1. In some embodiments, the biological sample is a blood sample. In some embodiments, the blood sample comprises circulating tumor DNA (ctDNA). In some embodiments, determining the number and distribution of the copies of amplified regions comprises performing a single primer extension using any one or more of SEQ ID NOs: 1-10 as a portion of the primer being extended. In some embodiments, determining the number and distribution of the copies of amplified regions comprises performing a single primer extension using SEQ ID NO: 1 as a portion of the primer being extended. In some embodiments, the sequence specific primer comprises a synthetic primer tag. In some embodiments, the sequence specific primer further comprises a universal primer sequence.

Some embodiments provided herein relate to methods of determining if a canine animal is likely to have cancer. In some embodiments, the methods include obtaining circulating cell free DNA (cfDNA) in a biological sample from a canine containing canine genomic sequences; amplifying SINE sequences and adjacent sequences from the canine genomic sequences to determine the number and distribution of SINE sequences in the canine genomic sequences; and determining whether the companion animal is likely to have cancer based on the number and distribution of the SINE sequences.

In some embodiments, the biological sample is a blood sample. In some embodiments, amplifying the SINE sequences is performed using a primer as set forth in any one of SEQ ID NOs: 1, 2, 3, 4, 5, 6, 7, 8, 9, or 10, or any combination or variant thereof. In some embodiments, the variant has a sequence identity of at least 75% to the sequence as set forth in any one of SEQ ID NOs: 1-10, such as 75%, 80%, 85%, 90%, 91%, 92%, 93,%, 94%, 95%, 96%, 97%, 98%, or 99%, or a sequence identity within a range defined by any two of the aforementioned values. In some embodiments, amplifying the SINE sequences comprises amplifying the SINE sequences using a primer comprising SEQ ID NO: 1.

Some embodiments provided herein relate to methods for profiling single nucleotide variants (SNVs) and copy number aberrations (CNAs) in a sample simultaneously, such as in a single assay. In some embodiments, the methods include adding various concentrations of short interspersed nuclear element (SINE) sequences to SNV panels. In some embodiments, SINE sequences are included in an amount ranging from about 0.001 nM to about 100 nM, such as 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 50, 60, 70, 80, 90, or 100 nM SINE sequences, or in an amount within a range defined by any two of the aforementioned values. In some embodiments, the combination of SNV panel and SINE sequences are added to a sample, and a SINE assay library preparation is performed, and sequenced.

Some embodiments provided herein relate to kits. In some embodiments, the kits are for determining cancer in a companion animal. In some embodiments, the kits include at least one sequence specific primer for amplifying cfDNA in a biological sample from a companion animal, wherein the at least one primer amplifies SINE repeat sequences and adjacent genomic sequences; and a polymerase for amplifying the primers.

In some embodiments, the kits further include blood collection tubes for collecting blood from a companion animal. In some embodiments, the at least one primer comprises the nucleotide sequence as set forth in any one of SEQ ID NOs: 1, 2, 3, 4, 5, 6, 7, 8, 9, or 10, or any combination or variant thereof. In some embodiments, the variant has a sequence identity of at least 75% to the sequence as set forth in any one of SEQ ID NOs: 1-10, such as 75%, 80%, 85%, 90%, 91%, 92%, 93,%, 94%, 95%, 96%, 97%, 98%, or 99%, or a sequence identity within a range defined by any two of the aforementioned values. In some embodiments, the at least one primer comprises the nucleotide sequence of SEQ ID NO: 1. In some embodiments, the at least one sequence specific primer comprises a synthetic primer tag. In some embodiments, the at least one sequence specific primer further comprises a universal primer sequence.

It should be realized that the genome-wide copy number aberration analysis described herein may be part of a larger diagnostic suite used to determine a companion animal's overall health. For example, the copy number aberration analysis may be used simultaneously or sequentially with other methods for detection, diagnosis, staging, screening, treatment, and management of cancer including additional genetic variance analysis. These procedures may be useful to detect a variety of cancers, including feline leukemia, squamous cell carcinoma, feline mammary cancer, mast cell tumors, bladder cancer, osteosarcoma, hemangiosarcoma or a variety of other cancers afflicting companion animals.

In alternative embodiments, copy number aberrations, including aneuploidies can be detected by amplifying interspersed repetitive nucleotide elements other than the SINE sequences. For example, aneuploidy can be detected by amplifying long terminal repeats that exist in the companion animal genome. One type of long terminal repeat present in companion animals are the Long Interspersed Nucleotide Elements (LINEs). These LINE sequences may be analyzed as described above, or may be detected by a variety of other known techniques. For example, in some embodiments, aneuploidy can be detected by any of the variety of methods disclosed in Patent Cooperation Treaty application publication number WO2013148496, the contents of which are incorporated herein by reference in their entirety. Those of ordinary skill in the art will be aware of other suitable methods for detecting aneuploidy chromosomes that contain LINES and SINE.

In some embodiments, the methods include obtaining or having obtained a biological sample from an animal that is suspected of having cancer. In some embodiments, the sample is a liquid biopsy sample, such as a blood sample. In some embodiments, the sample includes cfDNA. In some embodiments, the sample is provided in an amount of less than 10 mL, such as 10 mL, 9 mL, 8 mL, 7 mL, 6, mL, 5 mL, 4 mL, 3 mL 2 mL, 1 mL, 500 μL, 250 μL, 100 μL or an amount within a range defined by any two of the aforementioned values. In some embodiments, the sample includes DNA in an amount of less than or equal to 10 μg, such as 10 μg, 5 μg, 1 μg, 500 ng, 100 ng, 50 ng, 10 ng, 5 ng, 1 ng, 500 pg, 100 pg, 50 pg, 10 pg, 9, pg, 8 pg, 7 pg, 6 pg, 5 pg, 4 pg, 3 pg, 2 pg, or 1 pg, or in an amount within a range defined by any two of the aforementioned values. In some embodiments, the method includes purifying the DNA from the sample. Purifying the DNA may be accomplished using DNA purification techniques, including, for example extraction techniques, precipitations, chromatography, bead based methods, or commercially available kits for DNA purification.

In some embodiments, amplification products such as PCR or SBE products are sequenced to identify a presence of copy number aberrations or aneuploidy. Sequencing may include, for example, targeted low pass sequencing. In some embodiments, low pass sequencing can generate reads at a relatively low coverage. For example, the average coverage can be at least about 5×, 4×, 3×, 2×, 1×, 0.5× or less of the genome. The average coverages can be used to describe both the amplified regions of the genome or the whole genome. In some embodiments, low pass sequencing is used for measuring genome-wide genetic variation by variant calling across the whole genome.

In some embodiments, the presence of a copy number aberration in a sample is detected by using short interspersed nucleotide element (SINE) nucleotide sequence motifs as primers for amplicon sequencing. The motifs were identified using the MEME suite. The MEME suite is a set of motif-based sequence analysis tools. More information on these tools can be found on the Internet at meme-suite.org.

In some embodiments, the methods further include identifying an aneuploidy in the sample, which is one type of copy number aberration. In some embodiments, identifying the aneuploidy includes amplifying the purified DNA sample to look for repeated chromosomes or chromosomal fragments. It will be appreciated that any of the amplification methodologies described herein or generally known in the art can be utilized with universal or target-specific primers to amplify nucleic acids. Suitable methods for amplification include, but are not limited to, the polymerase chain reaction (PCR), strand displacement amplification (SDA), transcription mediated amplification (TMA) and nucleic acid sequence based amplification (NASBA). The above amplification methods can be employed to amplify one or more nucleic acids of interest. For example, PCR, including multiplex PCR, SDA, TMA, NASBA and the like can be utilized to amplify nucleic acids. In some embodiments, primers directed specifically to the nucleic acid of interest are included in the amplification reaction.

Definitions

The terms “cancer” and “cancerous” have their ordinary meaning as understood in light of the specification, and refer to or describe the physiological condition in animals that is typically characterized by unregulated cell growth. A “tumor” comprises one or more cancerous cells. There are several main types of cancer. Carcinoma is a cancer that originates from epithelial cells, for example skin cells or lining of intestinal tract. Sarcoma is a cancer that originates from mesenchymal cells, for example bone, cartilage, fat, muscle, blood vessels, or other connective or supportive tissue. Leukemia is a cancer that originates in hematopoietic cells, such as the bone marrow, and causes large numbers of abnormal blood cells to be produced and enter the blood. Lymphoma and multiple myeloma are cancers that originate in the lymphoid cells of lymph nodes. Central nervous system cancers are cancers that originate in the central nervous system and spinal cord.

As used herein, the term copy number aberration (CNA) means a change in the number of copies of a particular genetic sequence or component within an individual genome and can range from losses (deletions) of one or more copies of the genetic component to gains of numerous additional copies of the genetic component (amplifications). One type of CNA is an “aneuploidy”, which generally refers to an abnormal number of whole chromosomes. Typically, aneuploidy may result from a genetic imbalance resulting from cancer or other diseases. In some embodiments, aneuploidies results in either three (“trisomy”) or only one (“monosomy”) chromosome. In some embodiments, measuring aneuploidy may be used in the context of cancer diagnostics as described above.

As used herein, a “motif” has its ordinary meaning as understood in light of the specification, and refers to a nucleic acid sequence identified as being specific to a particular sequence. Motifs may include a specific nucleic acid sequence of less than about 160 base pairs, such as 150, 140, 130, 120, 110, 100, 90, 80, 70, 60, 50, 40, 30, 20, or 15 bp, or in an amount within a range defined by any two of the aforementioned values. In some embodiments, the motif includes a SINE motif having a nucleic acid sequence as set forth in any one of SEQ ID NOs: 1-10, or a nucleic acid sequence having a sequence identity of greater than 70% to any one of SEQ ID NOs: 1-10, such as 70%, 75%, 80%, 85%, 90%, 95%, 96%, 97%, 98%, or 99% sequence identify to any one of SEQ ID NOs: 1-10, or in an amount defined by any two of the aforementioned values. In some embodiments, the motif includes any fragment or subset of any one of SEQ ID NOs: 1-10.

As used herein, the phrase “allele” or “allelic variant” has its ordinary meaning as understood in light of the specification, and refers to a variant of a locus or gene. In some embodiments, a particular allele of a locus or gene is associated with a particular phenotype, for example, altered risk of developing a disease or condition, likelihood of progressing to a particular disease or condition stage, amenability to particular therapeutics, susceptibility to infection, immune function, etc.

As used herein, the term “amplification” has its ordinary meaning as understood in light of the specification, and refers to any methods known in the art for copying a target nucleic acid, thereby increasing the number of copies of a selected nucleic acid sequence. Amplification may be exponential or linear. A target nucleic acid may be either DNA or RNA. Typically, the sequences amplified in this manner form an “amplicon.” Amplification may be accomplished with various methods including, but not limited to, the polymerase chain reaction (“PCR”), transcription-based amplification, isothermal amplification, rolling circle amplification, etc. Amplification may be performed with relatively similar amount of each primer of a primer pair to generate a double stranded amplicon. However, asymmetric PCR may be used to amplify predominantly or exclusively a single stranded product as is well known in the art (e.g., Poddar et al. Molec. And Cell. Probes 14:25-32 (2000)). This can be achieved using each pair of primers by reducing the concentration of one primer significantly relative to the other primer of the pair (e.g., 100 fold difference).

Amplification by asymmetric PCR is generally linear. A skilled artisan will understand that different amplification methods may be used together.

As used herein, “amplicon” has its ordinary meaning as understood in light of the specification and refers to the nucleic acid sequence that will be amplified as well as the resulting nucleic acid polymer of an amplification reaction. An amplicon can be formed artificially, such as through polymerase chain reactions (PCR) or ligase chain reactions (LCR), or naturally through gene duplication.

As used herein, the term “short interspersed nuclear elements” (SINE) has its ordinary meaning as understood in light of the specification, and refers to non-autonomous, non-coding transposable elements (TEs) that are about 80 to 700 base pairs in length. The internal regions of SINEs originate from tRNA and remain highly conserved. As described herein, variations in the genome-wide copy number of SINE sequences in companion animals may be diagnostic for a variety of cancers.

As used herein, the term “companion animal” has its ordinary meaning as understood in light of the specification and includes dogs, cats, and horses and may also include any other domesticated animal normally maintained in or near the household of the owner or person who cares for such other domesticated animal. Examples of such additional companion animals may include rabbits, ferrets, pigs, gerbils, hamsters, chinchillas, rats, guinea pigs, horses, parrots, passerines, fowls, turtles, lizards, and snakes.

As used herein, the term “liquid biopsy” has its ordinary meaning as understood in light of the specification, and refers to the collection of a sample and the testing the sample, wherein the sample is non-solid biological tissue such as blood.

As used herein, the term “cfDNA” has its ordinary meaning as understood light of the specification, and refers to circulating cell free DNA, which includes DNA fragments released to the blood plasma. cfDNA can include circulating tumor deoxyribonucleic acid (ctDNA).

As used herein, the term “ctDNA” has its ordinary meaning as understood in light of the specification, and refers to circulating tumor DNA, which includes a tumor-derived fragmented DNA in the bloodstream that is not associated with cells.

As used herein, the terms “isolated,” “to isolate,” “isolation,” “purified,” “to purify,” “purification,” and grammatical equivalents thereof as used herein, unless specified otherwise, refer to the reduction in the amount of at least one contaminant (such as protein and/or nucleic acid sequence) from a sample or from a source (e.g., a cell) from which the material is isolated. Thus, purification results in an “enrichment,” for example, an increase in the amount of a desirable protein and/or nucleic acid sequence in the sample.

As used herein, the terms “amplify” or “amplified” “amplifying” as used in reference to a nucleic acid or nucleic acid reactions, refer to in vitro methods of making copies of a particular nucleic acid, such as a target nucleic acid, for example, by an embodiment of the present invention. Numerous methods of amplifying nucleic acids are known in the art, and amplification reactions include polymerase chain reactions, ligase chain reactions, strand displacement amplification reactions, rolling circle amplification reactions, multiple annealing and looping based amplification cycles (MALBAC), transcription-mediated amplification methods such as NASBA, loop mediated amplification methods (e.g., “LAMP” amplification using loop-forming sequences.

### EXAMPLES

Embodiments of the present invention are further defined in the following Examples. It should be understood that these Examples are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims. The disclosure of each reference set forth herein is incorporated herein by reference in its entirety, and for the disclosure referenced herein.

### Example 1

### Identifying Motifs

Using the MEME suite, motifs present in short interspaced nuclear element (SINE) sequences in the canine genome were identified. The canine genome used was the CanFam 3.1 dog genome from the Dog Genome Sequencing Consortium. This genome sequence can be found at GenBank assembly accession GCA_000002285.2. Several motifs in the canine genome were identified, including SEQ ID NOs: 1-10. The motifs were identified using SINE repeat masker sequences available through the Institute of Systems Biology (ISB) and found on the Internet at repeatmasker.org/species/canFam.html.

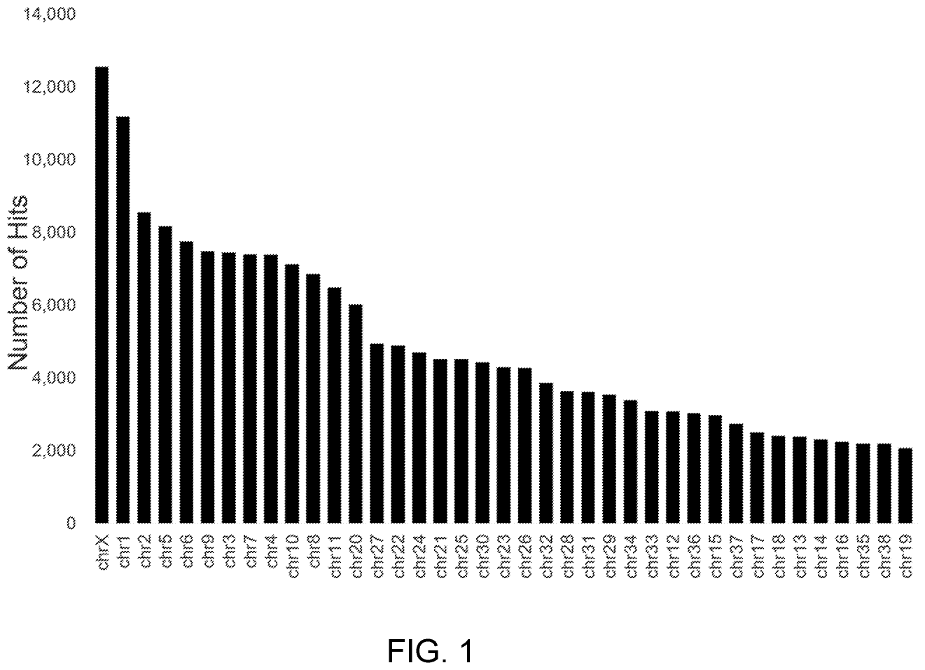

FIG. 1 depicts the number of times that SEQ ID NO: 1 (GAGCCTGCTTCTCCCTCTGCCTSTGTCTCT, where S is C or G) was found on canine chromosomes using targeted low-pass whole genome sequencing to capture genome wide cancer-specific aneuploidies. The motif set forth in SEQ ID NO: 1 was found at 192,301 chromosomal sites at 30 base pairs, with 100% sequence identity. The motif set forth in SEQ ID NO: 1 was also found at 313,238 sites at 15 base pairs and 100% sequence identity and 588,958 sites at 15 base pairs and more than 90% sequence identity. 150 BP sequences downstream of the start of SEQ ID NO: 1 from 171K sites were aggregated and aligned to the CanFam3.1 reference genome.

Alignment to the reference genome was done using tools such as BWA (bio-bwa.sourceforge.net). 150 base pairs downstream of the start of the motif resulted in 170k/171k sites with a mapping quality (MAPQ) score of greater than 55 and 1347 sites with a MAPQ score of less than 30. The MAPQ score is a probabilistic measure of the uniqueness of an alignment with a score greater than 55 predicting the alignment is unique. 100 base pairs downstream of the start of SEQ ID NO: 1 resulted in 101k/171k sites with a MAPQ score of greater than 55, 31k sites with a MAPQ score of less than 30, and 6k sites with a MAPQ score of 0.

### Example 2

### Experimental Protocol

A blood sample was taken from a dog, and circulating free DNA was isolated. The fragmented samples were end-repaired and A-tailed in a single reaction to create 3′ A overhangs. The DNA fragments were then ligated at their 5′ ends to adaptors containing a unique molecular index (UMI) consisting of a 12-base fully randomized sequence. This randomization provides 412 possible combinations of unique indexes per adapter and provides a unique barcode for each fragment.

A sequence specific primer having both the sequence of SEQ ID NO: 1 and a synthetic primer tag used for multiplexing, as well as a universal primer that is specific to the adapter, were then added to the mixture. The sequence specific primer of SEQ ID NO: 1 hybridizes specifically to SINE sequences. DNA Polymerase is added to the mixture to enrich sequences adjacent to where the SEQ ID NO: 1 primer hybridizes. After removing the SEQ ID NO: 1 primer from the mixture, a universal primer and a primer complementary to the primer tag are added to amplify the enriched sequence and generate a library competent for next-generation sequencing. Included on the second primer is a sample index sequence, a unique multiplex sequence identifier that can be used after sequencing to identify particular sequences. In one embodiment, the primer with the primer tag contains a next generation sequencing primer binding location.

### Example 3

### Performing Canine Copy Number Aberration Analysis, including Aneuploidy Analysis

The following example demonstrates an example of a method for performing Copy Number Aberration analysis, including aneuploidy analysis, on a sample obtained from a dog.

A blood sample is obtained from a dog. The blood sample is processed to isolate cell-free DNA (cfDNA). The cfDNA is fragmented, barcoded, amplified and extended using the Qiagen QIAseq kit and SINE motif of SEQ ID NO: 1.

Targeted low pass sequencing is performed on the amplification products to capture genome-wide cancer specific variants using an Illumina MiSeq Sequencing System (Illumina, San Diego, Calif.). Variants are identified by determining the copy number of the amplified regions anchored to SEQ ID NO: 1. Bioinformatics methods or determining the copy number are described in Patent Cooperation Treaty application publication number WO2013148496 listing Bert Vogelstein as the first inventor.

### Example 4

### Copy Number Aberration Analysis using SINE and sWGS

The following example demonstrates an example of performing copy number aberration (CNA) analysis using the SINE method as compared to shallow whole genome sequencing (sWGS).

Three canine samples with a confirmed diagnosis of cancer from both tumor tissue and cfDNA were obtained. The samples were analyzed to determine CNA profiles using SINE and sWGS assays. A total of 18 libraries were sequenced, one from each of the three canine samples for cfDNA, gDNA, and tissue DNA sample, each using both SINE and sWGS.

For the three cfDNA samples, two 20 ng aliquots were obtained per sample. A SINE assay library prep was performed on the first aliquot, and a sWGS library prep was performed on the second aliquot.

Similarly, for the three gDNA samples, two 20 ng aliquots were obtained per sample. A SINE assay library prep was performed on the first aliquot, and a sWGS library prep was performed on the second aliquot.

Finally, for the three tissue DNA samples, two 20 ng aliquots were obtained per sample. A SINE assay library prep was performed on the first aliquot, and a sWGS library prep was performed on the second aliquot.

All libraries were sequenced on NovaSeq system using an SP flowcell (2×150 bp), and evaluated via bioinformatics metrics. Table 1 provides the consensus variants for the three samples. All events are chromosome-level noted as partial. For CNA gains, copy number is three unless otherwise noted.

TABLE 1
Consensus Variant Summary

| Sample | CNA Gain | CNA Loss |
|---|---|---|
| 1 tissue | chrs 9 (partial), | chrs 11 (partial), 29 (partial), |
| | 13, 27 (partial) | X (partial) |
| 1 cfDNA | chr 13 | chrs 11 (partial), 29 (partial), |
| | | X (partial) |
| 2 tissue | none | none |
| 2 cfDNA | chrs 1, 4, 6, 9, 10, 12, 13 | chrs 2, 3, 5, 7, 8, 11, 14, 15, 16, |
| | (CN4), 19, 21, 23, 24, 25, | 17, 18, 20, 22, 27, 30 |
| | 26, 28, 29, 31, 32, 33, 34, | |
| | 35, 36, 37, 38, X | |
| 3 tissue | chrs 12, 18, 24, 30, 37 | chrs 5, 11, 15, 21, 25, 27, 38, X |
| 3 cfDNA | none | none |

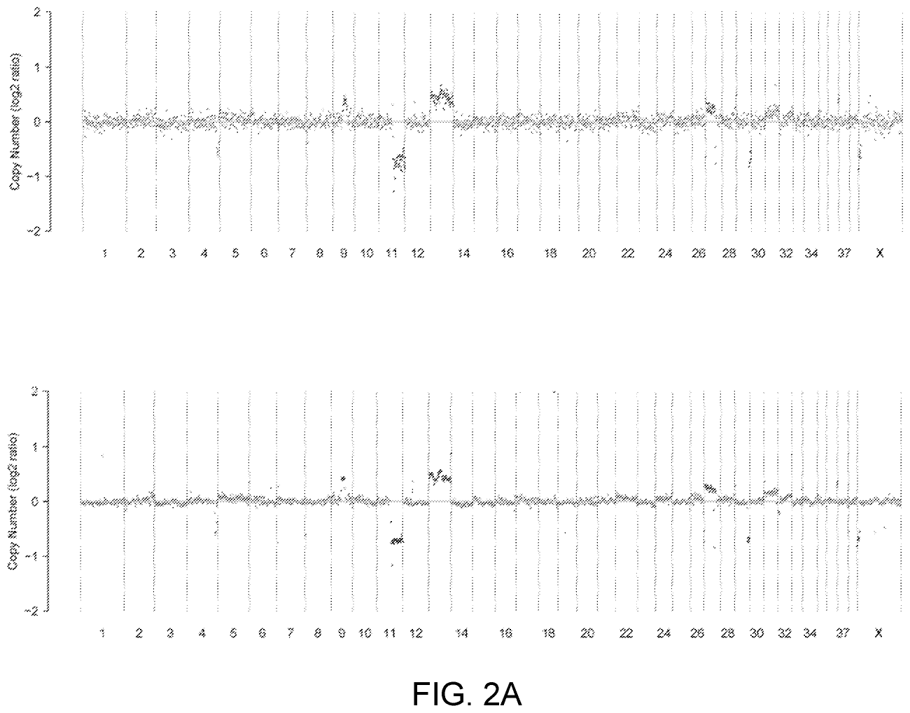

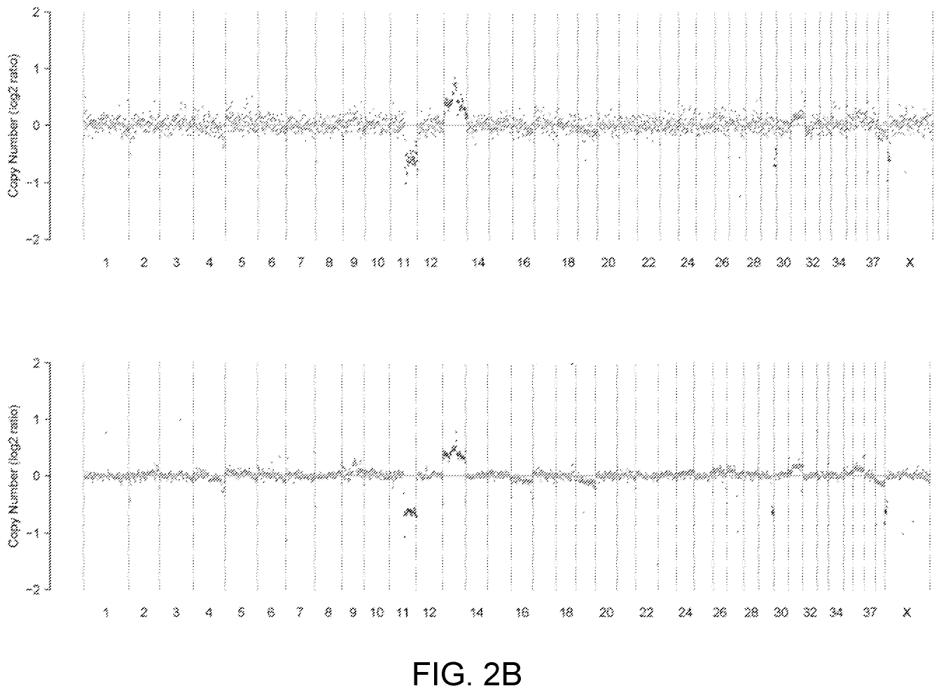

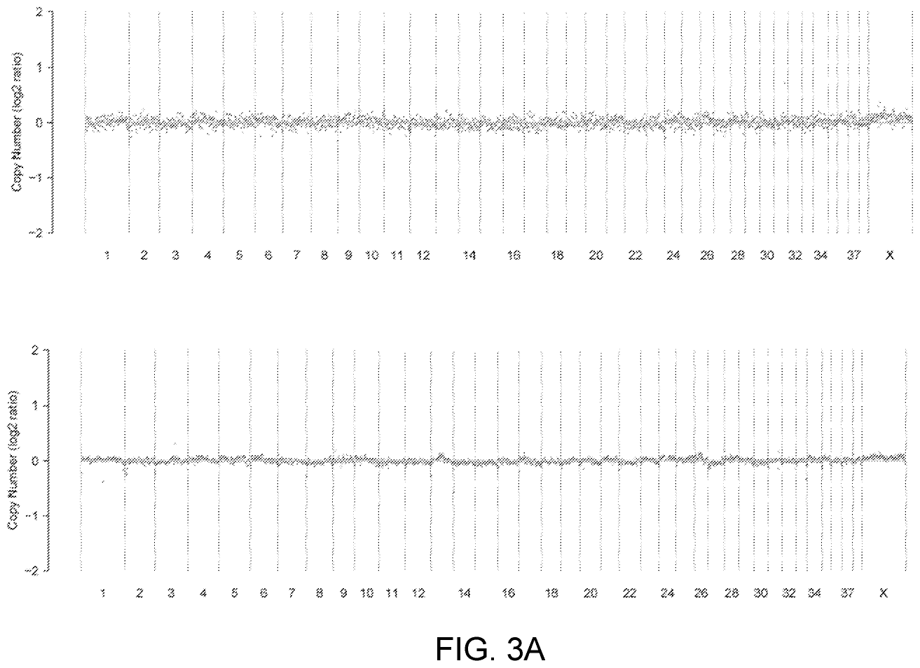

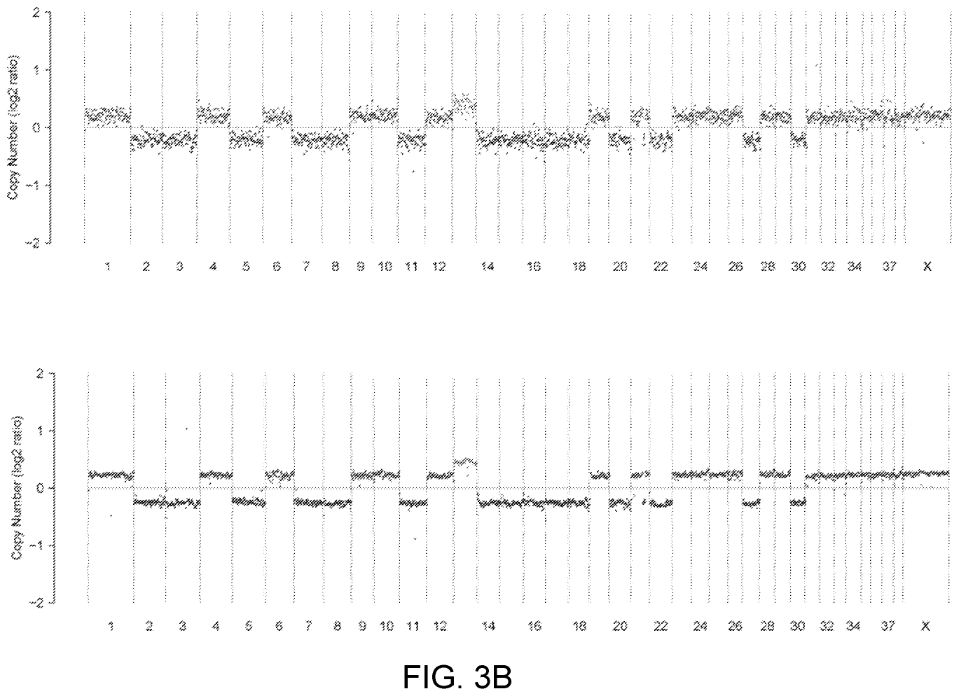

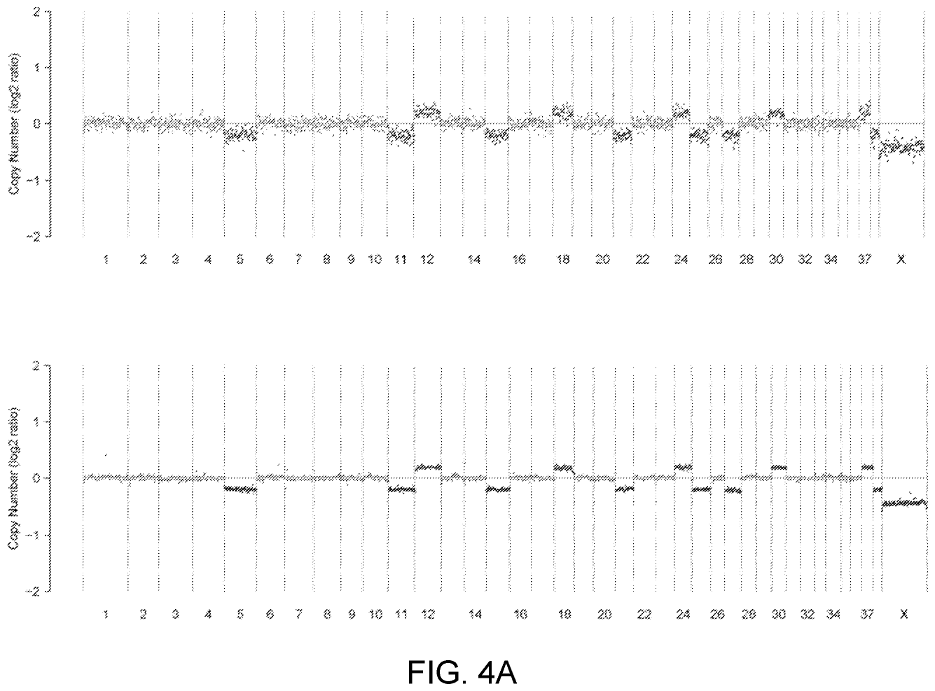

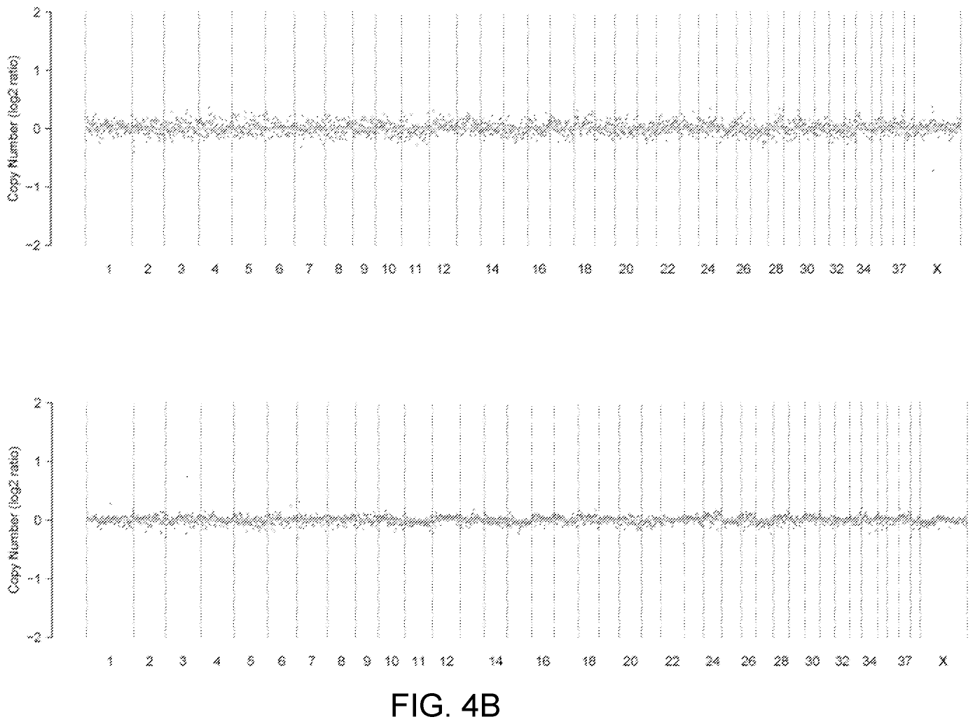

As shown in FIGS. 2A, 2B, 3A, 3B, 4A, and 4B, SINE and sWGS methods both deliver effectively equivalent CNA calls across all three samples and across both tumor and cfDNA specimen types. sWGS shows lower noise across bins, which is likely due to different collapsing/deduplicating methods. For example, as shown in FIGS. 2A and 2B, the sWGS profile (FIG. 2B) is tighter in terms of overall uniformity, due to the fact that the SINE file in FIG. 2A is UMI-collapsed, resulting in fewer reads being used. CNA calls do not necessarily agree between tissue and matched cfDNA samples, as shown in FIGS. 3A and 3B, and 4A and 4B. Thus, it is likely that tissue sample collected in FIG. 3A contained normal cells, rather than tumor cells, as no CNAs were detected in the tumor tissue. Further, as shown in FIG. 4B, no CNAs were detected in cfDNA, indicating that the cfDNA does not necessarily reflect faithfully the full heterogeneity of the individual's tumors.

### Example 5

### Evaluation of Single Nucleotide Variant and SINE Spike

The following example demonstrates an example of mixing a SINE primer and a single nucleotide variant (SNV) panel at various ratios. This example was designed to test the feasibility of profiling SNV and CNA simultaneously.

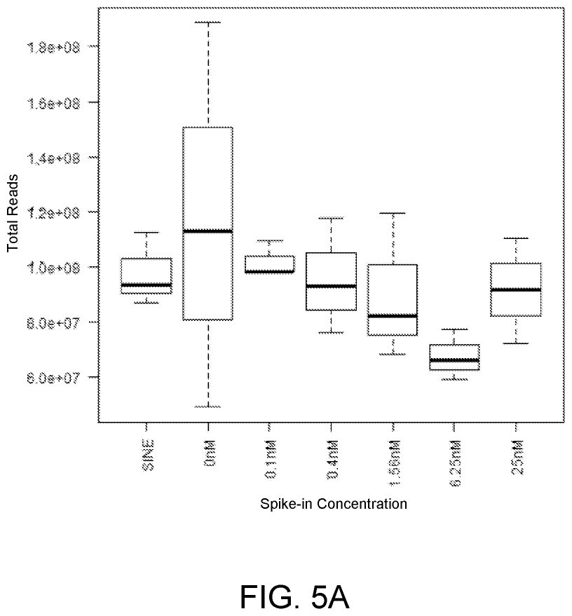

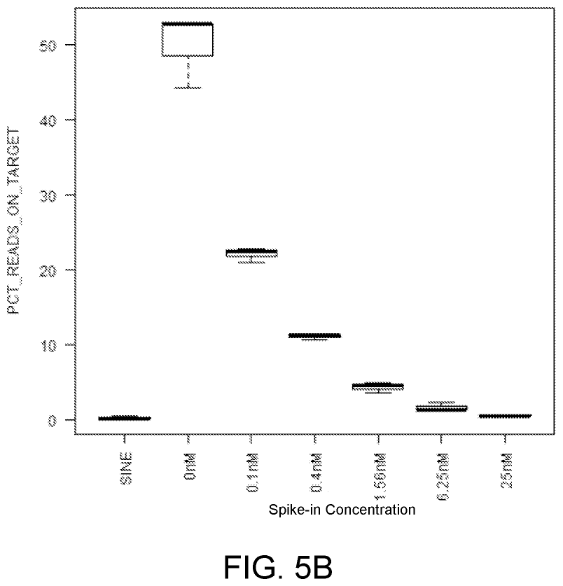

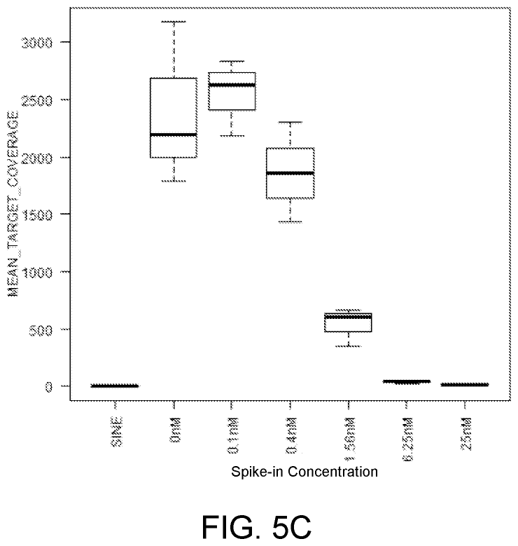

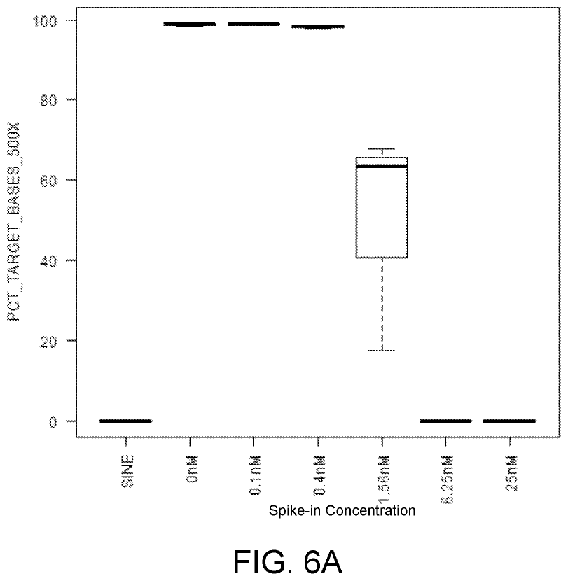

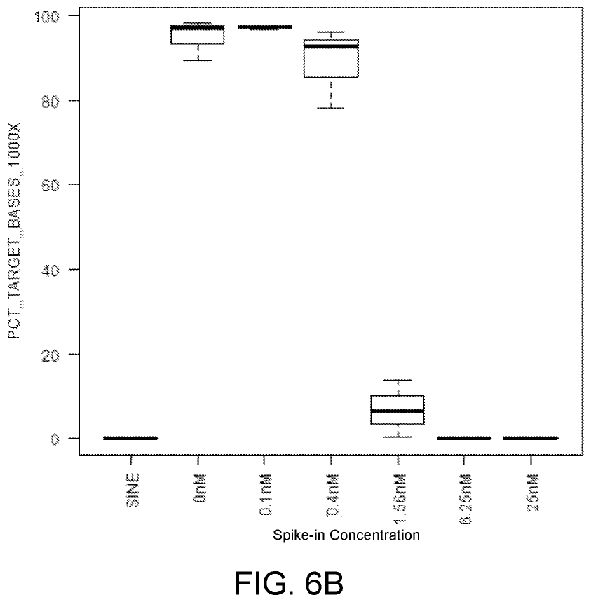

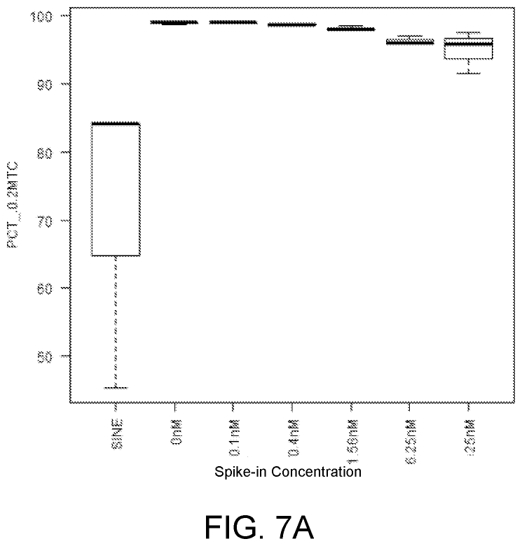

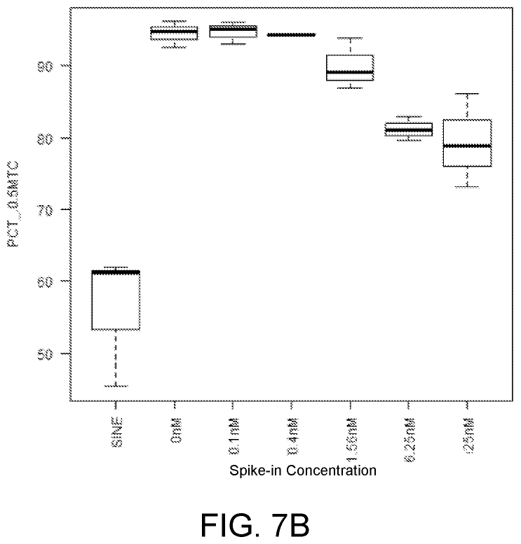

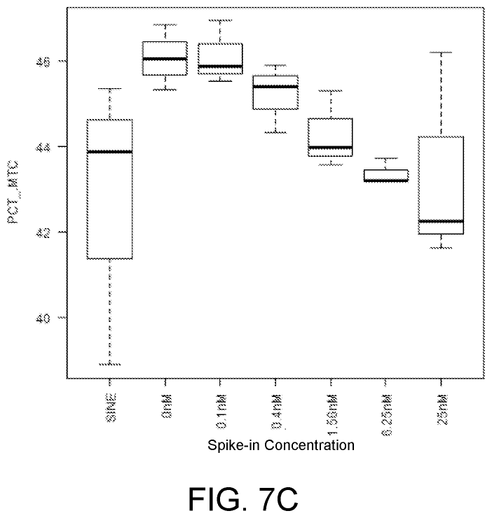

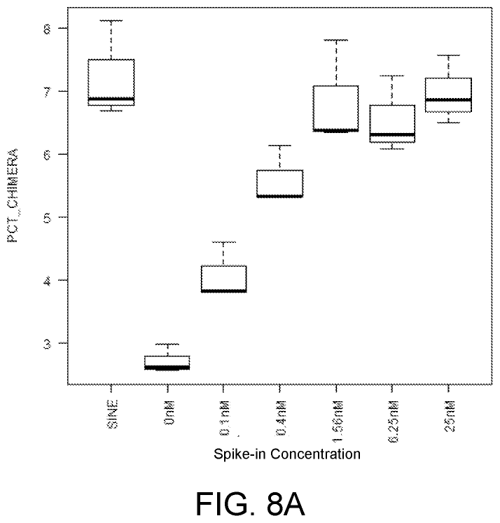

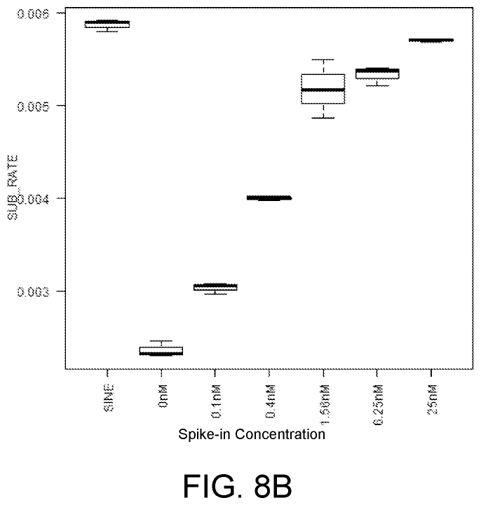

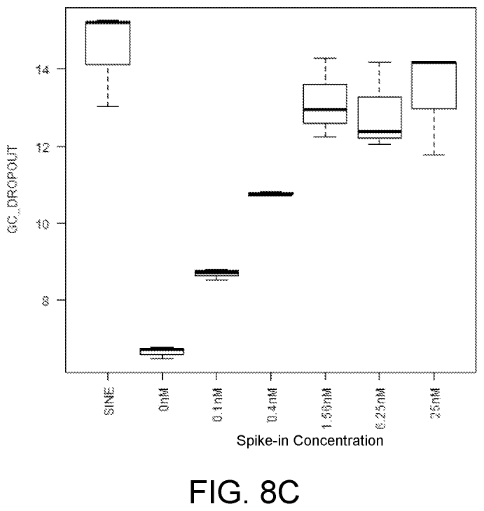

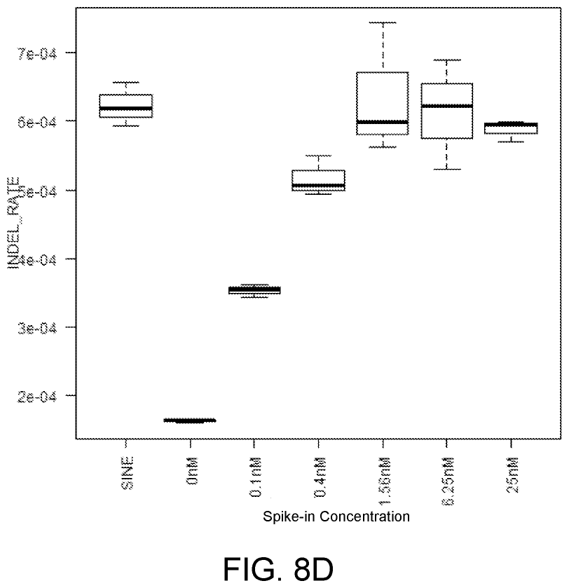

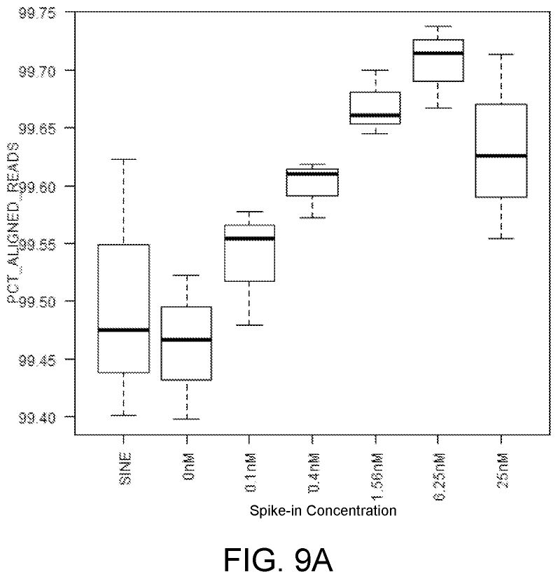

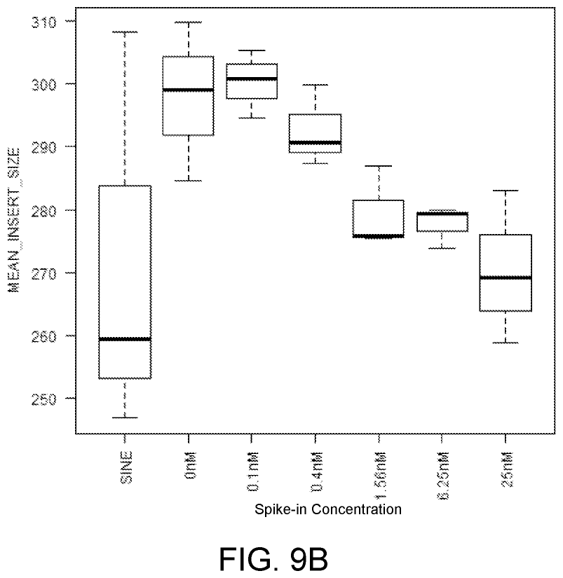

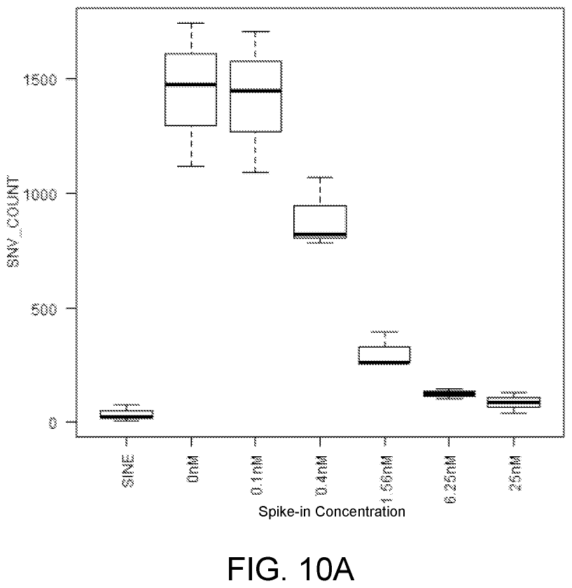

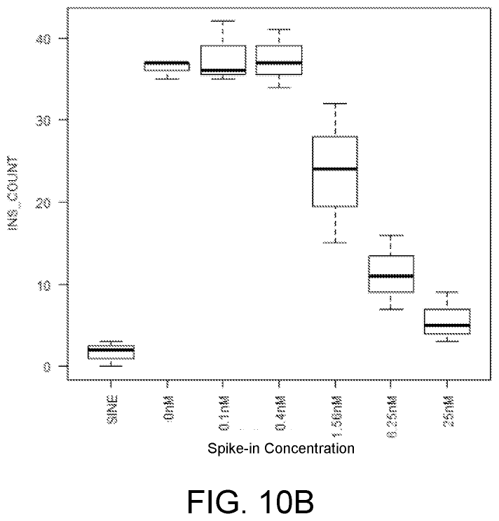

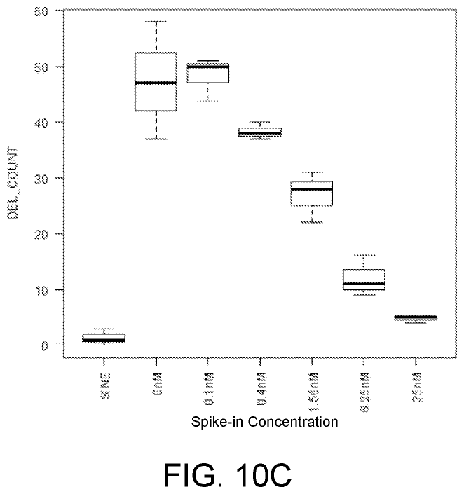

An SNV panel was obtained (QIASeq targeted panel analysis). The panel was added to various concentrations of SINE, including SINE alone (25 nM), SNV panel with 0 nM SINE, SNV panel with 0.1 nM SINE, SNV panel with 0.4 nM SINE, SNV panel with 1.56 nM SINE, SNV panel with 6.25 nM SINE, and SNV panel with 25 nM SINE. Samples and primer mixtures included each of the three canine samples as set forth in Example 4 (canine samples 1, 2, and 3), with SNV panel in combination with the SINE concentrations set forth above, or with SINE at 25 nM alone. For each sample, a SINE assay library prep was performed. All libraries were sequenced on NovaSeq using an SP flowcell (2×150 bp), and evaluated via bioinformatics metrics. Output metrics were combined across replicates and conditions and plots were generated using standard plotting calls. As shown in FIGS. 5A, 5B, 5C, 6A, 6B, 7A, 7B, 7C, 8A, 8B, 8C, 8D, 9A, 9B, 10A, 10B, and 10C, SINE primer spike-in into the SNV panel shows the ability to profile both SNV and CNA events simultaneously. The optimal spike-in concentration appears to be approximately 0.1 nM SINE, as this level resulted in nearly identical metrics and variant calling performance as the SNV panel alone, while still yielding a significant number of SINE reads (˜30M on average), that is sufficient for CNA calling.

As used herein, the section headings are for organizational purposes only and are not to be construed as limiting the described subject matter in any way. All literature and similar materials cited in this application, including but not limited to, patents, patent applications, articles, books, treatises, and interne web pages are expressly incorporated by reference in their entirety for any purpose, including the disclosures specifically referenced herein. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

As used in this specification and claims, the singular forms “a,” “an” and “the” include plural references unless the content clearly dictates otherwise.

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Although this invention has been disclosed in the context of certain embodiments and examples, those skilled in the art will understand that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes or embodiments of the disclosed invention. Thus, it is intended that the scope of the present invention herein disclosed should not be limited by the particular disclosed embodiments described above.

It should be understood, however, that this detailed description, while indicating preferred embodiments of the invention, is given by way of illustration only, since various changes and modifications within the spirit and scope of the invention will become apparent to those skilled in the art.

The terminology used in the description presented herein is not intended to be interpreted in any limited or restrictive manner. Rather, the terminology is simply being utilized in conjunction with a detailed description of embodiments of the systems, methods and related components. Furthermore, embodiments may comprise several novel features, no single one of which is solely responsible for its desirable attributes or is believed to be essential to practicing the inventions herein described.

What is claimed is:

1. A method of determining if a companion animal is likely to have cancer, comprising:
obtaining circulating cell free DNA (cfDNA) in a biological sample from a companion animal;
amplifying the cfDNA using a sequence specific primer that is derived from repeat elements present throughout the genome of the companion animal to obtain copies of the repeat element and adjacent genomic sequences;
determining the number and distribution of the copies of amplified regions in the cfDNA; and
comparing the number and distribution of copies of the amplified regions, including the adjacent genomic sequences, to one or more healthy animals to determine if the number and distribution of copies in the companion animal suspected of having cancer differs from the number of copies of the amplified regions in the one or more healthy animals, wherein a statistically significant difference indicates that the companion animal is highly likely to have cancer.

2. The method of claim 1, wherein the companion animal is a dog.

3. The method of claim 1, wherein the sequence specific primer is present in short interspersed nuclear element (SINE) sequences.

4. The method of claim 3, wherein the sequence specific primer has a nucleotide sequence of any one of SEQ ID NOs: 1-10, or a sequence having at least 90% sequence identity thereof.

5. The method of claim 3, wherein the sequence specific primer has a nucleotide sequence of SEQ ID NO: 1.

6. The method of claim 1, wherein the biological sample is a blood sample.

7. The method of claim 6, wherein the blood sample comprises circulating tumor DNA (ctDNA).

8. The method of claim 1, wherein determining the number and distribution of the copies of amplified regions comprises performing a single primer extension using any one or more of SEQ ID NOs: 1-10, or a sequence having at least 90% sequence identity thereof, as a portion of the primer being extended.

9. The method of claim 1, wherein determining the number and distribution of the copies of amplified regions comprises performing a single primer extension using SEQ ID NO: 1 as a portion of the primer being extended.

10. The method of claim 1, wherein the sequence specific primer comprises a synthetic primer tag.

11. The method of claim 10, wherein the sequence specific primer further comprises a universal primer sequence.

12. A method of determining if a canine animal is likely to have cancer, comprising:
obtaining circulating cell free DNA (cfDNA) in a biological sample from a canine containing canine genomic sequences;
amplifying short interspersed nuclear element (SINE) sequences and adjacent sequences from the canine genomic sequences to determine the number and distribution of SINE sequences in the canine genomic sequences; and
determining whether the companion animal is likely to have cancer based on the number and distribution of the SINE sequences.

13. The method of claim 12, wherein the biological sample is a blood sample.

14. The method of claim 12, wherein amplifying the SINE sequences comprises amplifying the SINE sequences using a primer comprising any one or more of SEQ ID NOs:
1-10, or a sequence having a sequence identity of at least 90% thereof.

15. The method of claim 12, wherein amplifying the SINE sequences comprises amplifying the SINE sequences using a primer comprising SEQ ID NO: 1.

16. The method of claim 12, further comprising determining single nucleotide variants by contacting the sample with a single nucleotide variant (SNV) panel and spike-in concentrations of SINE sequences.

17. A method of profiling single nucleotide variant (SNV) and copy number aberration (CNA) in a single assay, comprising:
obtaining circulating cell free DNA (cfDNA) in a biological sample from a canine containing canine genomic sequences;
contacting the sample with primers for an SNV and with primers for a CNA using short interspersed nuclear element (SINE) spike sequences; and
amplifying the SINE spike sequences;
thereby determining SNV and CNA in a single assay.

18. The method of claim 17, wherein the SINE spike sequences are present in an amount ranging from about 0.1 nM to about 25 nM.

19. A kit for determining cancer in a companion animal, comprising:
at least one sequence specific primer for amplifying cfDNA in a biological sample from a companion animal, wherein the at least one primer amplifies short interspersed nuclear element (SINE) repeat sequences and adjacent genomic sequences; and
a polymerase for amplifying the primers.

20. The kit of claim 19, further comprising blood collection tubes for collecting blood from a companion animal.

21. The kit of claim 19, wherein the at least one primer comprises the nucleotide sequence of any one or more of SEQ ID NOs: 1-10, or a sequence having a sequence identity of at least 90% thereof.

22. The kit of claim 19, wherein the at least one primer comprises the nucleotide sequence of SEQ ID NO: 1.

23. The kit of claim 19, wherein the at least one sequence specific primer comprises a synthetic primer tag.

24. The kit of claim 19, wherein the at least one sequence specific primer further comprises a universal primer sequence.
